(19) United States
(12) Patent Application Publication
(10) Pub. No.: US 2012/0116448 A1
(43) Pub. Date: May 10, 2012

(54) LENGTH-CONTROL SUTURE TECHNIQUE

(76) Inventor: Robert S. Reiffel, Scarsdale, NY (US)

(21) Appl. No.: 13/286,149

(22) Filed: Oct. 31, 2011

Related U.S. Application Data
(60) Provisional application No. 61/408,582, filed on Oct. 30, 2010.

(51) Int. Cl. A61B 17/04

(52) U.S. Cl. 606/223; 606/228

(57) ABSTRACT

A method of suturing a wound using a length-control technique. The method includes the step of placing a first pass of suture having barbs, with a first pre-attached needle, completely through the skin of the wound, from the undersurface to the outside, at a shallow angle just beyond one apex of the wound. The method also includes the step of placing the opposite end of the suture, with a second pre-attached needle, completely through the skin of the wound, from the undersurface to the outside, at a shallow angle just beyond the opposite apex of the wound. When the skin is pushed down on the suture, the suture pulls both apices towards the center of the wound to connect both apices to each other.

### CROSS REFERENCE TO RELATED APPLICATION

This application claims the benefit of U.S. Provisional Application No. 61/408,582, filed on Oct. 30, 2010, the entire contents of which are incorporated herein by reference.

### BACKGROUND OF THE INVENTION

1. Field of the Invention

The present invention generally relates to a length-control suture method, and more particularly to a new method of suturing that can reduce or prevent hypertrophic scars and “dog-ears” from forming during healing.

2. Related Art

An example of a fairly well known approach to suturing wounds is to use a common suture, in a zig-zag pattern from side to side across the wound to pull the sides together and allow them to heal. However, surgical and traumatic wounds tend to develop hypertrophic scarring when exposed to lengthwise stress. Therefore, one drawback to this method and other known methods is that they can result in thickening of the wound into an unsightly scar following suturing, such as a hypertrophic scar. In addition, when tissue is removed and the resultant wound is sutured together side-to-side, the ends of the wound (apices) tend to stick up in the air in a configuration referred to as a “dog-ear.”

There exists, therefore, a need to provide a novel suturing method that can prevent wounds from thickening into such an unsightly scar after suturing and to prevent or limit the size of the dog-ear that forms when tissue is excised.

### SUMMARY OF THE INVENTION

Problems such as the foregoing are overcome by a novel length-control suture method that can reduce or prevent hypertrophic scars and dog-ears from forming after the suturing is performed or during healing.

In more detail, the present invention in one embodiment provides a novel suture technique that involves the placement of a single suture directly from one end of a wound to the other, using a type of suture with barbs on it that, when put through tissue, cannot be then pulled backwards. The present invention can thereby prevent the body from making the wound thicken into an unsightly scar such as a hypertrophic scar or dog-ear, or at least reduce such scarring. Also by virtue of the features of the present invention, any scarring from surgery that does occur can be softer, paler, narrower, and with minimal puckering when compared with scarring from surgery not performed by this technique.

In still more detail, the present invention according to one aspect provides a method of suturing a wound using a length-control technique. The method includes the step of placing a first pass of suture having barbs, with a first pre-attached needle, completely through the skin of the wound, from the undersurface to the outside, at a shallow angle (e.g., approximately 15 degrees) just beyond one apex of the wound. The method also includes the step of placing the opposite end of the suture, with a second pre-attached needle, completely through the skin of the wound, from the undersurface to the outside, at a shallow angle (e.g., approximately 15 degrees) just beyond the opposite apex of the wound. When the skin is pushed down on the suture, the suture pulls both apices towards the center of the wound to connect both apices to each other.

The present invention according to another aspect provides a length-control suture method using a straight micro-point cutting taper needle. The method comprises (a) passing a single suture having barbs thereon directly from one end of a wound to the other end of the wound without holding any side-wall tissue in between, and (b) anchoring the suture to an underside of the dermis just beyond both apices of the wound, wherein the suture pulls both apices of the wound towards the center of the defect.

The present invention according to another aspect provides a needle and suture kit for use in a length-control suture method. The kit includes a needle that is double-ended, straight, tapered, and has a micro-point cutting tip on each end. The kit also includes a suture having multiple barbs thereon, wherein the barbs are spaced close together so as to effect a large amount of tissue contact over a shorter distance.

### BRIEF DESCRIPTION OF THE DRAWINGS

Further features and advantages of the present invention will be more readily understood from a detailed description of the exemplary embodiments taken in conjunction with the following figures in which:

FIG. 1 (comprised of FIGS. 1A and 1B) is a Table showing the strength retention profile and other characteristics of various sutures.

FIG. 2 shows a surgical technique of the present invention according to one embodiment.

FIG. 3 shows a device for use with the surgical technique of the present invention.

The invention will next be described in connection with certain exemplary embodiments; however, it should be clear to those skilled in the art that various modifications, additions, and subtractions can be made without departing from the spirit or scope of the claims.

### DETAILED DESCRIPTION OF THE PREFERRED EMBODIMENTS

Part One: Hypertrophic Scarring

Much has been written about the cause and prevention of hypertrophic scarring after dermal injury. Numerous studies have been performed as to the causes of such scarring at the cellular and biochemical levels. Whatever the cause at the microscopic level, clinical modifications of healing tissue at the macroscopic level have yielded variable success. Treatment methods studied include topical modalities such as silicone gel sheeting and zinc, external compression via elastic garments or splints, surgical excision, corticosteroid injection, cryotherapy, chemotherapy, ultrasound, laser treatment, irradiation, and the application of paper tape.

In an effort to elucidate the cause of hypertrophic scarring, many studies have focused on the suture material itself, comparing absorbable sutures such as polyglycolic acid (PGA) or catgut with non-absorbable materials such as silk, nylon, or polypropylene. Additional studies compared absorbable sutures with other absorbable sutures (polyglactin and polydioxanone), and non-absorbable sutures with other non-absorbable sutures (polybutester and nylon). The majority of these studies report minimal difference in scar outcome with respect to the suture material used and, instead, attribute the largest variation in hypertrophy rates to incision location and orientation.

It is well known that surgical and other traumatic wounds typically develop hypertrophic scarring when exposed to lengthwise stress during the initial phases of healing. If a wound lies parallel to a natural skin flexion crease, hypertrophic scarring will generally not occur. However, wounds that cross flexion creases tend to thicken. In many anatomic regions, stretching of the skin occurs in several different directions, making it difficult or impossible to place the incision precisely in a skin fold. The use of paper tape, applied longitudinally over such wounds, has been shown to limit the lengthwise stretching, with a resultant reduction in the occurrence of hypertrophic scarring. Similarly, prevention of facial muscle movement (and therefore facial wound stretching) by means of subjacent botulinum toxin injection has been shown to improve scar outcome in facial wounds.

Suture technique has been examined as well, although not as extensively as suture material. Both intracutaneous butterfly sutures and modified vertical mattress sutures have been used to improve scar outcome. However, with these methods, as with other conventional suture techniques, the pattern of suture placement has never been one that would impart a strict limit to the amount of scar elongation allowed, as the sutures were always placed in a side-to-side, zig-zag, or ladder-rung pattern.

Since all such suture patterns, whether placed in a simple interrupted, continuous intradermal, or locked intradermal pattern, do allow some accordion-type stretch of the wound, the inventor of the present application has found that an altogether different technique of suture placement, with a suture of particular characteristics, is necessary to impart a strict limit on wound elongation. Just as paper tape applied to the external surface of a healing scar can prevent virtually all longitudinal stretching and has been shown to eliminate or materially diminish the tendency for scar hypertrophy, a suture technique applied from the undersurface would need to impart the same absolute limitation on stretching from underneath so as to minimize scar hypertrophy.

Therefore, with such goals in mind, the applicant devised a new and novel suture technique: the “length-control” suture technique (LCS), which uses a certain placement of suture and a suture having certain characteristics (e.g., having barbs on it) that, when put through tissue, cannot be then pulled backwards. One type of suture suitable with the present invention is a Quill® suture, which is currently used only to suture the edges of a wound together. That said, as described in more detail below, the Quill® needle is curved and cutting, while even more preferably a needle for use with the present invention is straight and only micro-point cutting, meaning only cutting at the tip.

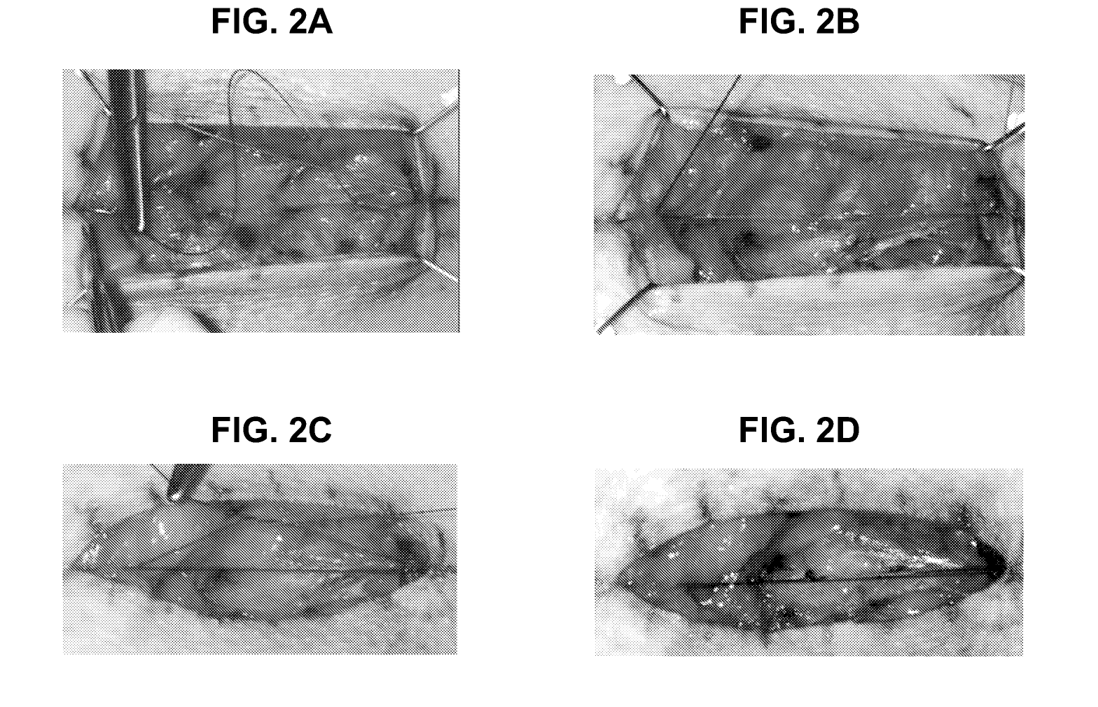

The technique of the present invention, being an end to end technique, is different from conventional side to side, ladder, or zigzag techniques. With the technique of the present invention, suture is passed directly from one end of the wound to the other, in a closed loop if it's standard suture, or in a single strand if the suture is barbed, without holding any tissue in between. See FIGS. 2a-b. In FIG. 2a (top left), the first pass of the suture has been placed securely into the undersurface of the dermis just beyond one apex of the wound. In FIG. 2b (top right), the next bite of the suture grabs the deep portion of the dermis just beyond the opposite apex of the wound. In FIG. 2c (bottom left), the suture is firmly anchored to the undersurface of the dermis just beyond both apices of the wound and pulls them towards the center of the defect; accordingly, the suture connects the two apices of the wound to each other. If barbs are not being used on the suture, a knot is tied, and as the knot is tied the two apices are pulled slightly towards the center. However, if barbs are used on the suture as described herein (although no barbs are actually shown in FIG. 2), such a knot is not needed.

In FIG. 2d (bottom right), as an added measure to insulate the length-control suture from the surface, the knot (if used) is placed at the center of the defect, where it can be securely covered by an additional two-layer closure, thereby minimizing the likelihood of exposure and extrusion. Even the deeper layer of this additional two-layer closure is placed completely superficial to the entirety of the length-control suture.

Part Two: Dog Ears

This technique may also be used to prevent or reduce the formation of dog ears and to ensure that wounds over a convex surface do not assume a concave configuration once healed. When tissue is excised, the resulting wound assumes a fusiform shape. When those defects are closed using standard techniques, the center of such wounds is the tightest, while the apices tend to be looser and can bulge up from the surrounding surface in a shape known as a dog ear.

By using the length-control suture technique (LCS) of the present invention, wounds can be protected from lengthwise stress, and therefore protected from the forces that lead to the formation of dog ears.

Surgical and traumatic wounds tend to develop hypertrophic scarring when exposed to lengthwise stress. The length-control suture (LCS) technique of the present invention, in which a barbed suture material is passed in a straight line beneath the wound and anchored to the underside of the dermis beyond each apex (e.g., into a portion of the dermis that is not part of the wound; or “normal” skin), thereby pulling the apices inwards, can protect wounds from such forces, and the formation of dog ears can be limited or prevented. When employed, this technique can result in scars that are reliably flatter, thinner, softer, and more supple within 6 months. Within weeks the scar can exhibit signs of softening.

An important factor in the length-control suture technique (LCS) of the present invention is the tension under which the suture is placed. If a non-barbed suture in a closed loop is used, when it is first tied there should be a slight inward pull on the apices of the wound. As the deep dermal layer is then placed, one will note that the apices have become even more taught. This indicates that the length-control suture is already beginning to perform its function: preventing wound elongation. This inward pull will typically persist for weeks to months and is a key to the length-control suture's effectiveness. As the scar matures, the pull will eventually disappear.

The tendency towards hypertrophic scarring may continue for a number of months after dermal injury. Therefore, the tensile strength of the length-control suture material should be maintained for this extended time interval. While a non-absorbable suture could be used, there would always be the possibility of late extrusion.

This technique may also be used to prevent or reduce the formation of dog ears and to ensure that wounds over a convex surface do not assume a concave configuration once healed.

The present invention is therefore a length-control suture technique that is a novel skin closure technique. This technique serves to prevent or reduce hypertrophic scarring and dog ears by intrinsically minimizing the lengthwise strength on an incision during healing. The technique involves controlling the length of wounds by suturing the two apices together without including any side-wall tissue. The suture, itself, is a barbed suture with a micro-point cutting taper needle that allows the technique to be applied faster and simpler without the need for tying a knot in the center and with the added benefit of being able to adjust the tension on the wound at the end, once it is already closed, just by pulling up on the ends of the suture after they have exited the skin.

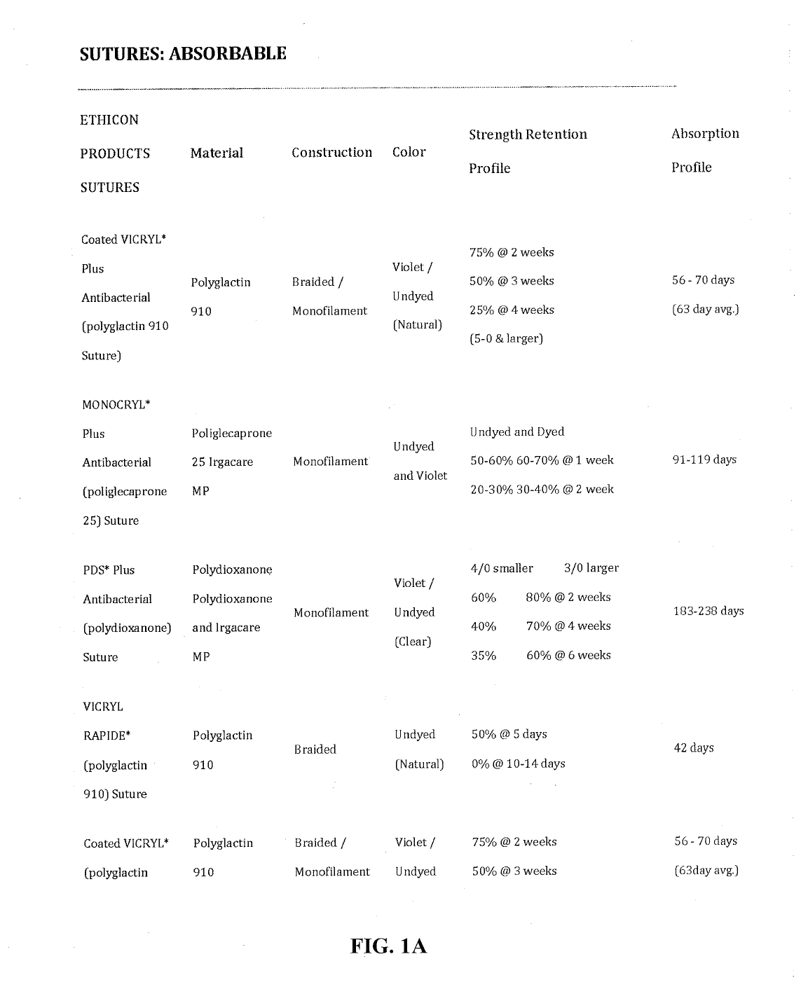

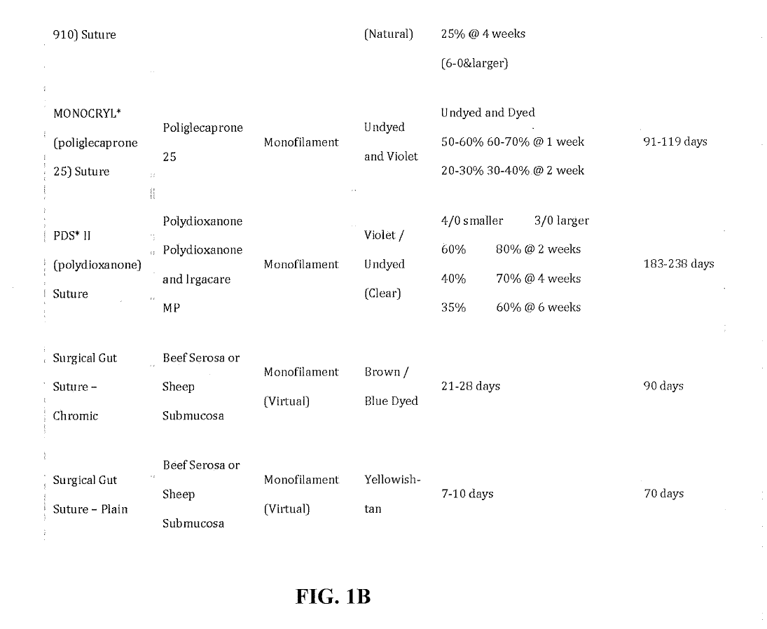

As shown in the Table of FIG. 1, various absorbable sutures maintain their tensile strength for different amounts of time. Therefore, when Panacryl® was developed, it was evident that this material could provide the necessary characteristics to function as a length-control suture: its tensile strength is maintained for approximately 6 months and it has the advantage that it will, eventually, disappear. Thus, Panacryl® was a suture chosen in an early version of this technique.

Of course, being a braided suture, Panacryl® had the possible disadvantage of eventual “spitting.” In fact, because of such untoward events, it was recalled as a “free” suture by the manufacturer in 2006 and is now only available attached to suture anchors. Early on in this series, Panacryl® did “spit” on one occasion, but in this instance it was used not only for the length-control suture, but also for closure of the subcutaneous tissues and deep dermis during revision of a sternotomy scar. Since then, Panacryl® was used only for the length-control suture, and did not present any additional problems. As it was no longer available, once existing supplies were exhausted, a change was made to PDS II®, with no significant change in results. However, the inventor of the present application found that the early version of the LCS technique, using Panacryl® or PDSII®, could be improved upon by using a suture with barbs on it that, when put through tissue, could not be thin pulled backwards. The Quill® suture was one such type of suture that improved upon the earlier version of the LCS technique of the present invention. One model of Quill® suture suitable for use with the present invention, as an example, is RA-1013Q.

Another important factor in the length-control suture technique of the present invention is the type of needle that is used therewith. The dermis is very tough material. For a needle to pass easily through the dermis, it must be a cutting needle. A cross-section of a cutting needle is a triangle with sharp (cutting) edges. It passes easily through dermis, but makes a big hole. A tapered needle, on the other hand, just spreads the tissue a little. It does not cut a big hole. It works well for sewing muscle and other soft tissues, but is hard to push through the skin.

A micro-point cutting needle as used with the technique of the present invention is a triangle only at the very tip—just to get the cutting started. Most of it is round in cross section. That is important because, if the hole were too large, the barbs on the suture would not grab the tissue. The needle for use with the technique of the present invention is preferably straight, not curved. The standard needle for sewing skin is curved, because it goes into the skin on each side and then back out. Such needle must be curved to follow that wavy path into and then back out of each side edge. While a curved needle is suitable to use with the technique of the present invention, it would not be the most preferred needle to use with the technique of the present invention. The suture of the present invention takes a very gentle, straight, glide path from the depths of the wound, gradually emerging out of the skin at some distance beyond each end of the wound. Such long, gentle ascent allows more barbs to grab the tissue on the way out, giving it the anchoring strength it needs. The suture technique of the present invention does not go through the sides of the wound at all. In addition, having the hole (made by the needle) be as small as possible causes the tissue to grab the barbs more securely.

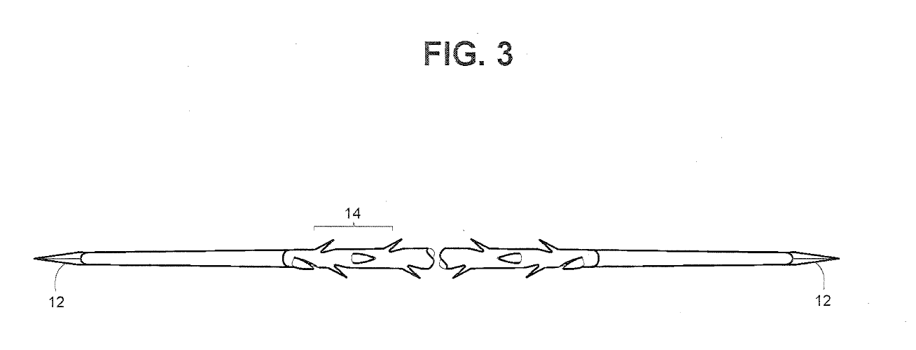

FIG. 3 shows an illustration of a preferred needle or device 10 of the present invention to be used with the LCS technique. The device 10 should be double-ended, meaning the same type of needle at each end. (While FIG. 3 only shows one end of the needle for simplicity sake, the opposite end of the needle would simply be a mirror image; that is, the needle is the same at the other end with minor-image barbs, i.e., with barbs pointing away from the cutting tip.) The needle should be a long, taper-cutting needle for end-to-end suturing to allow more barbs to grab the tissue. In more detail, the device 10 has a cutting tip 12 on each end and multiple barbs 14 on each end spaced fairly close together to allow a large amount of tissue contact over a shorter distance. The needle tapers and is wider at the base than near the tip. The type of suture material should be long-lasting absorbable monofilament. Of course, FIG. 3 is just one example embodiment, and the present invention is not to be limited by details shown therein; for example, the number of barbs or the size or shape of the barbs may be changed.

The process of scar hypertrophy (one of the issues that the technique of the present invention, and therefore the suture, is designed to prevent) generally peaks about 6 weeks after surgery. Therefore, preferably, the suture would maintain its strength for at least that interval. More preferably, the suture would maintain its strength for more than 6 weeks, e.g., for approximately 2-3 months, and most preferably for approximately 6 months. Suture made out of a monofilament can be barbed.

The effect of the technique of the present invention with respect to reducing or preventing dog-ears will now be further discussed. As the technique of the present invention further evolved, it became apparent to the inventor that the inward pull of the length-control suture could provide an added benefit even in wounds that were not likely to develop hypertrophy: control of “dog-ears.” If one observes the surface contour of a fresh defect after excision of a skin lesion before any sutures are placed, the topography of the area is relatively flat. However, once the deeper layer of skin sutures (i.e., deep dermal sutures) are placed, dog-ears appear at either apex. Dog-ears can be minimized if the skin incision is extended, with the apices made more tapered. Alternatively, dog-ears can be revised a number of months later, if necessary. However, these methods require either a longer scar or a second procedure. If the defect lies over a relatively convex anatomical feature, such as a cheekbone or a shoulder, the problem becomes magnified, due to the natural tendency of a sutured defect to assume a concave configuration. Under such circumstances, even a longer scar or an additional procedure may not suffice to restore the proper convex shape.

By using a length-control suture as disclosed in the present invention, dog-ears can be pulled into the center, thereby minimizing their appearance. Moreover, if the length-control suture is placed under slightly greater tension, a convex shape can even be created. If one is unsure as to how much tension should be applied, the length-control suture can be left protruding from the wound (usually near one apex) and tied after the deep dermal sutures are placed. It can be critically important, however, to push the knot of the suture down beneath the skin surface to prevent late extrusion. With the use of the barbed suture, since there is no knot and the suture protrudes from the skin beyond each apex, the tension can be tightened after the wound is closed merely by pulling up on the suture and pushing down on the skin before the excess, protruding, suture is cut off below the skin surface.

For such primary procedures, when only control of convexity and associated dog-ears are the goals, the length-control suture material can be polyglactin or a similar material. Obviously, for wounds on the face or nose, the suture material chosen for the length-control suture should not be 2-0 in caliber. Rather, a suture of 4-0, 5-0, or even 6-0 absorbable material suffices. Consideration must be given, however, to the Strength Retention Profile data to ensure that the suture can retain its strength during the hypertrophic healing phase of the wound in question.

As can be seen above, the length-control suture technique of the present invention is useful for a wide variety of both surgical and traumatic wounds on the face, trunk, and extremities. When employed, this technique can results in scars that are flat, thin, and supple within 6 months. Furthermore, the length-control suture allows for a single-stage procedure with minimal dog-ear formation and a scar of limited length, even over convex surfaces.

While various embodiments of the present invention have been described above, it should be understood that they have been presented by way of example, and not limitation. It will be apparent to persons skilled in the relevant art(s) that various changes in form and detail can be made therein without departing from the spirit and scope of the present invention. Thus, the present invention should not be limited by any of the above-described exemplary embodiments, but should be defined only in accordance with the following claims and their equivalents.

What is claimed is:

1. A method of suturing a wound using a length-control technique, comprising the steps of:
placing a first pass of suture having barbs, with a first pre-attached needle, completely through the skin of the wound, from the undersurface to the outside, at a shallow angle just beyond one apex of the wound; and
placing the opposite end of the suture, with a second pre-attached needle, completely through the skin of the wound, from the undersurface to the outside, at a shallow angle just beyond the opposite apex of the wound;
wherein when the skin is pushed down on the suture, the suture pulls both apices towards the center of the wound to connect both apices to each other.

2. The method of claim 1, wherein the method is performed using a straight micro-point cutting taper needle.

3. The method of claim 1, wherein the method is performed using a curved cutting needle.

4. The method of claim 1, further comprising the step of initially leaving the barbed suture protruding from the wound near one apex, and subsequently pulling the suture and tying the suture after the suture has exited the skin.

5. The method of claim 1, wherein the shallow angle is approximately 15 degrees.

6. A length-control suture method using a straight micro-point cutting taper needle, comprising:
passing a single suture having barbs thereon directly from one end of a wound to the other end of the wound without holding any side-wall tissue in between; and
anchoring the suture to an underside of the dermis just beyond both apices of the wound, wherein the suture pulls both apices of the wound towards the center of the defect.

7. The method of claim 6, further comprising the step of initially leaving the barbed suture protruding from the wound near one apex, and subsequently pulling the suture and tying the suture after the suture has exited the skin.

8. A needle and suture kit for use in a length-control suture method, comprising:
a needle that is double-ended, straight, tapered, and has a micro-point cutting tip on each end; and
a suture having multiple barbs thereon, wherein the barbs are spaced close together so as to effect a large amount of tissue contact over a shorter distance.

9. The kit of claim 8, wherein the suture is made of a long-lasting absorbable monofilament material.

10. The kit of claim 8, wherein the suture has a tensile strength such that the suture maintains its strength for at least approximately 6 weeks after surgery.

11. The kit of claim 8, wherein the suture has a tensile strength such that the suture maintains its strength for at least approximately 3 months after surgery.

12. The kit of claim 8, wherein the suture has a tensile strength such that the suture maintains its strength for at least approximately 6 months after surgery.
